(19) United States
(12) Patent Application Publication
(10) Pub. No.: US 2008/0208092 A1
(43) Pub. Date: Aug. 28, 2008

(54) Shoulder brace traction system

(76) Inventor: Thomas M. Sawa, Mississauga (CA)

Correspondence Address: DENNISON ASSOCIATES, 133 RICHMOND STREET WEST, SUITE 301, TORONTO, ON, M5H 2L7, omitted

(21) Appl. No.: 11/710,566

(22) Filed: Feb. 26, 2007

(51) Int. Cl. A61F 5/00

(52) U.S. Cl. 602/20; 602/19

(57) ABSTRACT

A shoulder brace traction system uses a torso fitting part to be worn by a user in combination with an upper arm fitting part to be worn by the user. The upper arm fitting part includes an extension overlay that extends and covers the shoulder cap of the user. The extension overly includes front and rear shoulder straps connected to an intermediate portion of the extension layer. These shoulder straps connect with and/or on the torso fitting part on front and rear portions of the shoulder. The extension includes two further straps adjacent the shoulder cap that engage front and rear central anchors on the torso fitting part. The brace traction system is effective for many athletes and is cost effective to manufacture.

### FIELD OF THE INVENTION

The present invention relates to a shoulder brace traction system for use by athletes to provide additional stability to an injured shoulder area and/or to assist in the prevention of injuries.

### BACKGROUND OF THE INVENTION

It is now widely recognized that an active life style is an important factor to an individual's health. Many people are continuing to participate in recreational and competitive sports as they age and to continue to participate even where minor injuries have occurred.

My earlier injury reduction and stabilizing harness, as disclosed in U.S. Pat. No. 4,735,198, has proven to be very effective for many athletes having shoulder injuries that require protection to continue to participate without excessive risk. This system provides an excellent alternative to the accepted prior practice of taping that requires experienced trainers or doctors, etc. to provide an effective taping of a shoulder. My system allows the player to basically wear the device and does not require the skill of others to stabilize and protect the shoulder of the athlete.

My pending United States Application US2006/0167395 provides a robust system to meet the demands of serious and/or paid athletes. Many of the stretch components used in my earlier design have been replaced with non stretch components and a series of control or limiting straps are also provided.

My earlier systems provided excellent protection of the shoulder joint, however, my most recent device is relatively sophisticated and often exceeds the requirements of an individual competing in a sport for exercise and recreational purposes. Thus, both of these systems exceed the requirements and cost sensitivity of the typical “weekend warrior”. Furthermore, these systems, due to the complexity thereof, are more expensive, and less demanding athletes have significant reluctance to pay for such a brace system as a prevention device.

There remains a need to provide an effective system that is easy to use and adjust while providing effective reinforcing or stabilizing of the shoulder area.

### SUMMARY OF THE INVENTION

A shoulder brace traction system according to the present invention comprises a torso fitting part, an upper arm fitting part, an extension overlay connected to the upper arm fitting part with a series of straps connectable to the torso fitting part to reinforce the shoulder area. The torso fitting part basically provides a series of anchor positions for engaging various straps which extend from the upper arm fitting part. The upper arm fitting part includes a cuff hold portion that generally engages the arm of the user slightly below and across the bicep to maintain the cuff in a position on the arm generally below the bicep. This cuff includes the ability to stretch to accommodate normal arm function but is generally held against sliding along the length of the upper arm.

The cuff portion has the extension overlay joined to the cuff along an upper edge and extends over the shoulder cap of the user. The underside of the extension overlay includes a pair of shoulder straps, namely a front shoulder strap and a rear shoulder strap. These shoulder straps extend from the extension overlay at a midway point and extends upwardly and diagonally such that the front shoulder strap engages a shoulder anchor member of the torso brace system and the rear shoulder strap engages a shoulder anchor member below the shoulder cap. The overlay partially covers these shoulder straps and extends across the shoulder cap of the user. The extension overlay includes a front strap and a rear strap with the front strap extending to a central torso front anchor of the torso fitting part and the rear strap extending to a central torso rear anchor of the torso fitting part. The extension overlay, the front and rear shoulder straps, as well as the front and rear shoulder straps are preferably of a stretch material such as a neoprene type material or other neoprene composite material similar to existing brace systems that use a stretch composite material.

With a brace system, there is a desire to retain initial heat to partially relax or allow the muscles to stretch during initial exercise as well as the need to allow heat to pass through the system during maximum heat generation. Typically the brace materials are of a composite design where heat build up and moisture retention are the more difficult problems. These composite type materials typically do have heat retention properties and the material can be varied, according to the particular requirements to control heat and moisture build up.

In a preferred aspect of the invention, a shoulder brace traction system uses a stretchable fabric for the torso fitting part which is tight fitting and includes various anchor portions. These anchor portions are preferably one component of a Velcro™ fastening system or other type fast release fastening system. The front shoulder strap and the rear shoulder strap, as well as the front overlay strap and the rear overlay strap, all have an engaging portion to secure the straps to the anchors on the torso fitting part.

The torso fitting part is preferably of a fabric type material that promotes wicking of the moisture away from the skin of the user.

In a further aspect of the invention, the torso fitting part is connected to the upper arm fitting part by a fabric type material positioned below the pair of shoulder straps to act as a separating layer between the extension overlay and the straps relative to the user's skin. This arrangement reduces skin irritation.

With the present system, a user can easily put on the shoulder brace traction system with the various straps released from their anchor points. The pair of shoulder straps can then be positioned to engage the shoulder anchor, whereafter the front and rear overlay straps can engage the anchors on the torso fitting part. In this way, the system is easily adjusted for use by the individual.

### BRIEF DESCRIPTION OF THE DRAWINGS

Preferred embodiments of the invention are shown in the drawings, wherein:

FIG. 1 is a front view of a user with the shoulder brace traction system in an adjusted system;

FIG. 2 is a front view similar to FIG. 1 with the arm of the user extending outwardly to one side;

FIG. 3 is a front view of the should brace traction system showing the various straps in a release position;

FIG. 4 is a front view similar to FIG. 3 with two of these straps secured;

FIG. 5 is a front view with the two remaining straps in a secured position;

FIG. 6 is a top view of the secured should brace traction system;

FIG. 7 is a partial perspective front view of the secured shoulder brace traction system;

FIG. 8 is a partial rear perspective view of the shoulder brace traction system;

FIG. 9 is a side view of the shoulder brace traction system in a secured position; and

FIG. 10 is a partial rear perspective view of the shoulder brace traction system.

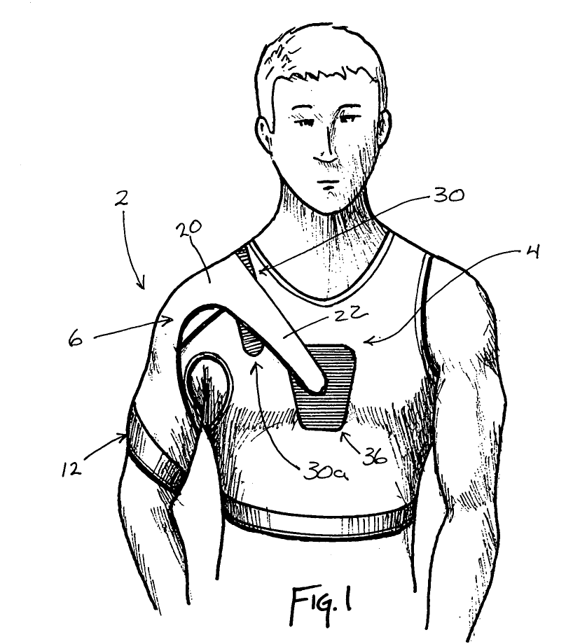

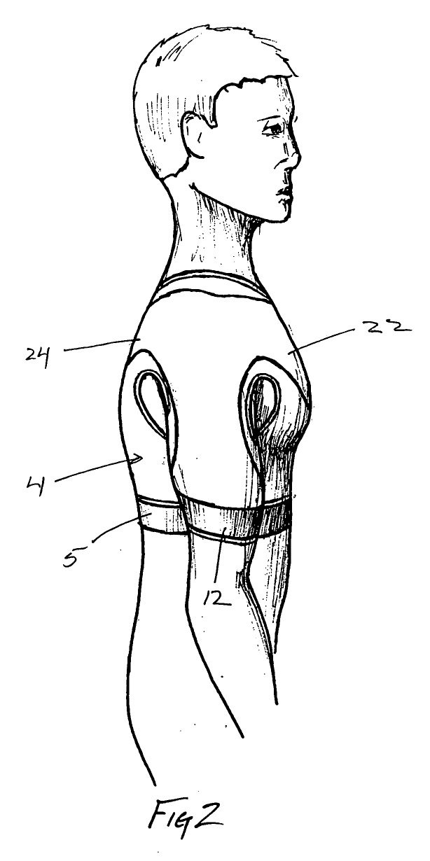

### DETAILED DESCRIPTION OF THE PREFERRED EMBODIMENTS

The shoulder brace traction system 2 shown in the various Figures includes a torso fitting part 4 in combination with an upper arm fitting part 6. This upper arm fitting part includes a cuff hold portion 12 securable below the user's bicep and extending partially around the user's bicep. The purpose of the torso fitting part 4 is to provide a support surface for the shoulder anchor member 30 that extends across the user's shoulder and provides a front securement segment 30a below the shoulder and a rear securement segment 30b below and on a rear surface of the shoulder. The torso fitting part 4 also includes the central torso front anchor 36 and the central torso rear anchor 38. Thus the torso fitting part 4 provides a member which is preferably of a resilient stretch material that closely fits the torso of the user. On this torso fitting part the various anchors (30a, 30b, 36 and 38) for the straps are provided in positions that are desirable for reinforcing and protecting the shoulder joint. Preferably, the torso fitting part is of a fabric type material that promotes wicking of moisture through the fabric and away from the user's skin. This particular material will be selected for providing the necessary support for the anchors as well as for the comfort of the user.

The cuff hold portion 12 is designed to engage the user's arm essentially below the bicep and has sufficient engagement with the arm to avoid sliding movement of the cuff portion upwardly along the upper arm. Typically, the cuff portion will be of resilient material to accommodate the flexing of the arm during use. The cuff portion can be a continuous cuff or a split cuff to allow adjustment thereof. The cuff portion can also be a strap or a wrap type arrangement to allow the user to appropriately secure the cuff portion about his arm. Thus the torso fitting part and the cuff hold portion basically provide reference anchor points for a series of straps that extend across the shoulder and provide reinforcing and stabilizing of the shoulder.

Extension overlay 20 is joined with an upper edge of the cuff holder portion 10 and extends upwardly and over the shoulder cap of the user. The underside of the extension overlay 20 includes a pair of shoulder straps, namely a front shoulder strap 8, and a rear shoulder strap 10. These shoulder straps engage the extension overlay generally at a central portion thereof, either side of the arm. Preferably, a stitched portion indicated as 42 (See FIG. 10), is provided on the extension 20 in a central portion, below the shoulder. This stitched portion can be an inverted “V” shape and extends downwardly and outwardly towards, and either side of the bicep. This provides additional reinforcing and a line of securement for the front and rear shoulder straps.

The front and rear shoulder straps 8, 10 extend diagonally upwardly and either frontwardly or rearwardly to engage the shoulder anchor member 30 on the torso fitting part 4. The shoulder anchor member 30 extends across the shoulder of the user and extends downwardly to the front and the rear of the user. As seen, the front and rear shoulder straps do not extend over the top of the shoulder but extend to either side of the top of the shoulder. These straps are releasably securable with the shoulder anchor member 30 by means of a quick fastening arrangement such as VELCRO or other similar fastening arrangements.

The main point is that these straps can easily engage the shoulder anchor portion 30 and retain engagement therewith. These straps are of a stretch material typically a composite layered material and includes a stretch body such as a neoprene type material. Many of these materials include a neoprene central portion with a stretch fabric either side thereof. As can be appreciated, these materials can include a series of punched holes or other arrangements for reducing the thermal retention characteristics. These straps could also be made of other materials such as a web material. The ability for the straps to stretch is desirable, however, it is also possible to use the stretch characteristics of the extension overlay 20.

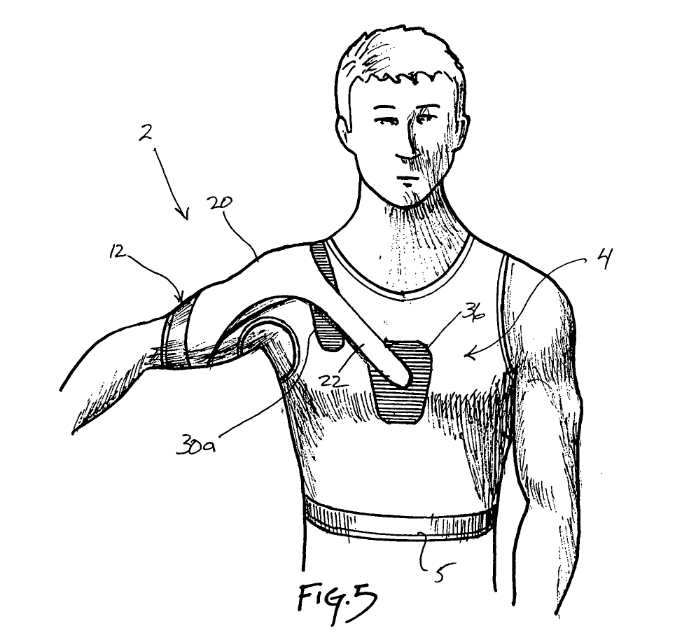

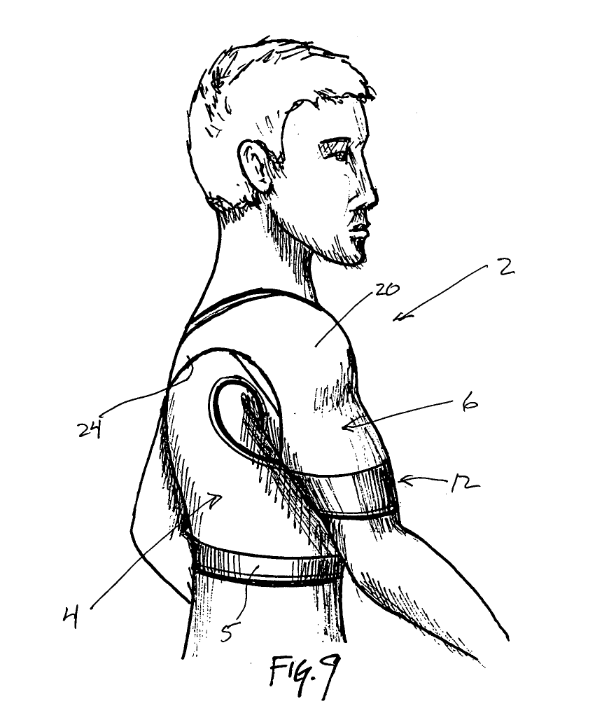

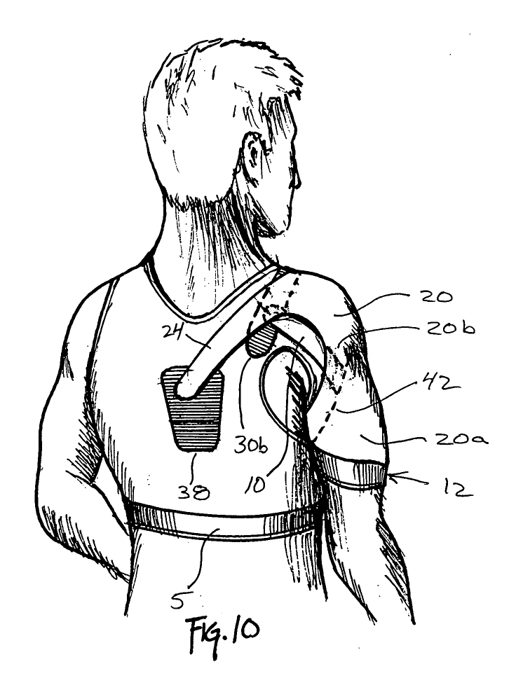

The extension overlay 20 preferably covers the shoulder cap of the user whereafter the front and rear overlay straps 22 and 24 are secured. As shown, the front and rear overlay straps are of a significantly longer length than the front and rear shoulder straps 8 and 10. The front overlay strap 22 extends from the shoulder cap downwardly and diagonally to the central torso front anchor 36 as evident in FIG. 5. As clearly shown in FIG. 8, the rear overlay strap 24 extends from the shoulder cap area downwardly and diagonally to the central torso rear anchor 38.

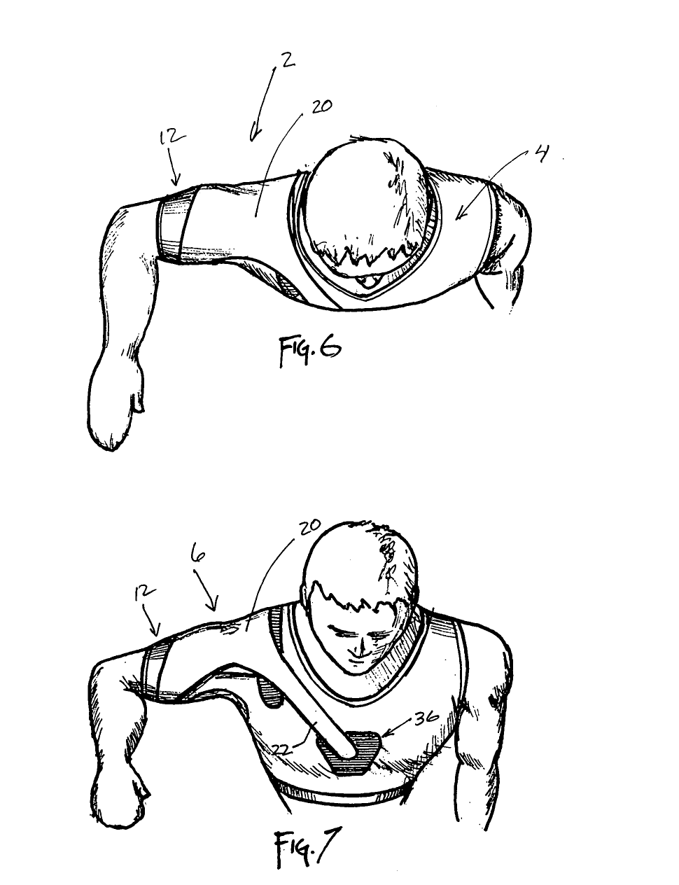

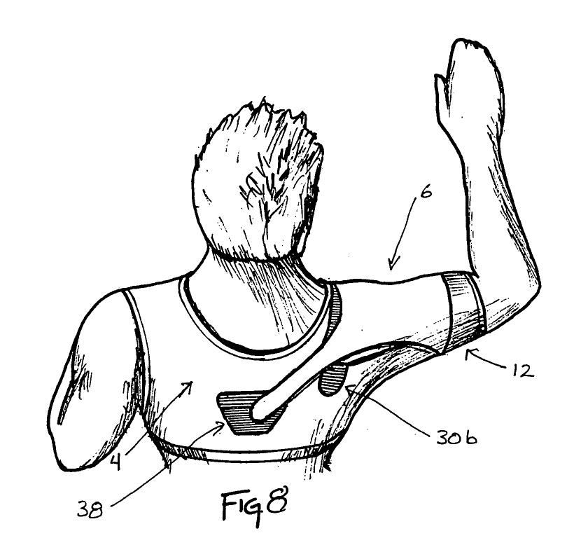

As shown in FIGS. 6, 7 and 8, the extension overlay 20 includes a first portion 20a that serves to join the cuff hold portion 12 with the reinforced stitched area 42. This reinforced area 42 of the overlay 20 is at an intermediate point and is located below the shoulder joint of the user. As shown in FIG. 10, the front and rear straps extend generally across the shoulder and below the shoulder to the shoulder anchor member 30.

A second portion of the overlay 20 is shown as 20b and is the portion of the overlay beyond the stitched reinforcement 42. This further extension of the overlay includes the front and rear overlay straps 22 and 24, either side of the shoulder cap. It is also positioned over the shoulder joint and allows the downward and diagonal type connection either side of the shoulder to the anchor portions 36 and 38.

With the above arrangement, a simplified shoulder brace traction system is realized that is easily used by an athlete. The system uses a series of straps which reinforce the shoulder area while still allowing good use of the arm. The system does not extend beneath the arm pit of the user primarily for comfort. Similarly, the extension overlay 20 partially wraps around the arm of the user but curves inwardly and is of a reduced width to avoid any irritation with the armpit area. Preferably, the fabric of the torso fitting part 4 also extends across the shoulder of the user and down the arm to provide a separation layer between the arm of the user and the reinforcing straps. As can be appreciated, the overlay 20 and the various straps are under tension and it is desirable to reduce friction or irritation between the skin of the user and the brace system. Furthermore, the system is preferably one piece where the cuff hold portion and the torso fitting part are connected. It is possible to have these as separate components. With two components, the torso fitting part can extend partially down the arm such that the straps are separated from the direct contact with the skin of the user.

Other arrangements for providing convenience of use and comfort are also possible.

The torso fitting part preferably includes a bottom grip tension member 5. This merely further secures the torso fitting part on the body of the user and opposes the tendency for the front and rear overlay straps to pull the torso fitting portion upwardly.

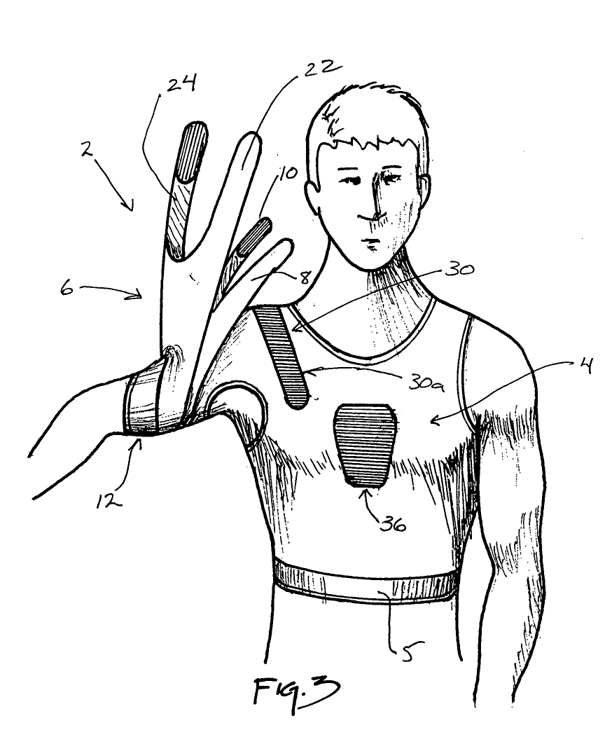

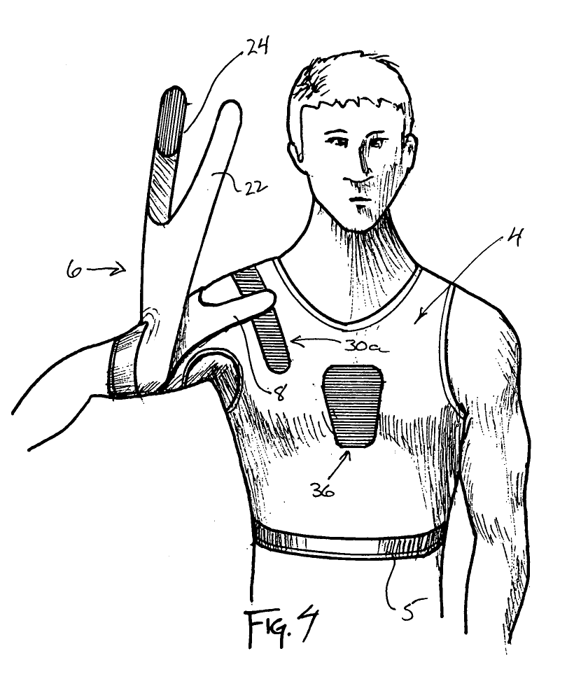

The securement of the various straps to the torso fitting part are demonstrated in FIGS. 3, 4 and 5.

Although various preferred embodiments of the present invention have been described herein in detail, it will be appreciated by those skilled in the art, that variations may be made thereto without departing from the spirit of the invention or the scope of the appended claims.

The embodiments of the invention in which an exclusive property or privilege is claimed are defined as follows:

1. A shoulder brace traction system comprising
a torso fitting part;
an upper arm fitting part having a cuff holding portion for securement below a bicep and extending partially thereacross;
an extension overlay portion joined to said cuff holding portion with a free end for placement above a shoulder cap;
said extension overlay between said free end and said cuff portion including an intermediate portion with upward and diagonally extending front and rear shoulder straps;
said torso fitting part including a shoulder anchor portion for releasably engaging and holding said front and rear shoulder straps in a tensioned condition;
said extending overlay at said free end including a front overlay strap and a rear overlay strap with each strap securable to said torso fitting part;
said torso fitting part including a central torso front anchor at a central portion of said torso fitting part and a rear anchor portion at a rear central portion of said torso fitting part;
said front overlay strap being securable to said front anchor and said rear overlay strap being securable to said rear anchor in a diagonal manner across said torso fitting member to maintain said overlay extension in a tensioned state during use of said shoulder brace traction system.

2. A shoulder brace traction system as claimed in claim 1 wherein said front and rear overlay straps are positioned beyond and spaced from said front and rear shoulder straps.

3. A shoulder brace traction system as claimed in claim 2 wherein said extension overlay and said front and rear overlay straps are integral and made of a composite stretch reinforcing material.

4. A shoulder brace traction system as claimed in claim 5 wherein said composite stretch reinforcing material includes a neoprene layer.

5. A shoulder brace traction system as claimed in claim 4 wherein said torso fitting portion is of a stretch fabric material designed to wick moisture away from the skin of a user.

6. A shoulder brace traction system as claimed in claim 5 wherein said cuff portion includes a fixed connection to said torso fitting portion.

7. A shoulder brace traction system as claimed in claim wherein said overlay is of a size to at least substantially cover said front and rear shoulder straps when said front and rear overlay straps are secured to said torso fitting portion and said system is in use.

9. A system as claimed in claim 8 wherein said front and rear shoulder straps are secured to an inverted ‘V’ shaped reinforced area of said extension overlay.

10. A system as claimed in claim 9 wherein said front and rear overlay straps are at least twice as long as said front and rear shoulder straps.

11. A system as claimed in claim 10 wherein said cuff holding portion, said extension overlay and said straps include an intermediate layer of a neoprene type material.
